SEQUENCE LISTING (1 sequence)

SEQ ID NO: 1 - DNA, 44 bp, Glycine max
taatttctactaagtgtagatgaacccttgagagaggcttcttc

(19) United States
(12) Patent Application Publication
(10) Pub. No.: US 2024/0327852 A1
(43) Pub. Date: Oct. 3, 2024

(54) METHODS OF TRANSFORMATION

(71) Applicant: SYNGENTA CROP PROTECTION AG, Basel (CH)

(72) Inventors: Heng Zhong, Research Triangle Park, NC (US); Changbao Li, Research Triangle Park, NC (US)

(73) Assignee: SYNGENTA CROP PROTECTION AG, Basel (CH)

(21) Appl. No.: 17/772,754

(22) PCT Filed: Nov. 24, 2020
(86) PCT No.: PCT/US2020/061901

§ 371 Date: Apr. 28, 2022

Related U.S. Application Data
(60) Provisional application No. 62/940,270, filed on Nov. 26, 2019.
(60) Provisional application No. 63/018,612, filed on May 1, 2020.

(51) Int. Cl. C12N 15/82

(52) U.S. Cl. CPC C12N 15/8205; C12N 15/8209; C12N 15/8213

(57) ABSTRACT

The disclosure relates to methods of transformation and related compositions. In some aspects, the methods comprise wounding at least part of a region of an embryo axis comprising an epicotyl, a shoot apical meristem, and cotyledonary node, or the corresponding region in a monocot embryo axis, to produce a wounded explant, and contacting the wounded explant with a heterologous polynucleotide. In some aspects, the methods further comprise on or more selection steps which may be done in planta.

### RELATED APPLICATIONS

This application claims priority from provisional applications 62/940,270 filed Nov. 26, 2019 and 63/018,612 filed May 1, 2020 incorporated by reference in their entirety herein.

### FIELD OF THE INVENTION

The invention relates to compositions and methods for transformation of plants.

### BACKGROUND

Conventional transformation methods are generally time-consuming and inefficient, and some elite lines have very low transformation efficiency using such conventional methods. Transformation is important for generation of transgenic plants and for genome editing of plants. There remains a need for more efficient, high-throughput, and less genotype-dependent methods of transformation.

### SUMMARY

The disclosure relates to methods of transformation. As described herein, methods were developed that involved transforming a wounded seed explant and utilizing one or more in planta selection steps. The methods described herein are useful, e.g., to introduce heterologous nucleic acids or proteins into plant cells for genome editing and transgenic plant generation. Such methods may increase efficiency, increase high-throughput capability, decrease chimerism and/or decrease genotype-dependency of transformation compared to conventional methods.

In some aspects, the disclosure provides a method, comprising: a) providing an explant of a seed, wherein the explant comprises an embryo axis and a cotyledon; b) wounding at least part of a region of the embryo axis to produce a wounded explant, wherein if the seed is a dicot seed then the region comprises an epicotyl, a shoot apical meristem, and cotyledonary node and if the seed is a monocot seed then the region comprises a coleoptile, a shoot apical meristem, a leaf primordia, and a leaf axillary region, and c) contacting the wounded explant with a heterologous polynucleotide and/or heterologous protein under conditions where the heterologous polynucleotide and/or heterologous protein enters the wounded explant. In some embodiments, the seed is an imbibed seed. In some embodiments, the seed has been imbibed in a liquid medium, optionally for up to 48 hours. In some embodiments, the explant is produced by removing a seed coat from the seed. In some embodiments, the wounding is performed by a method comprising cutting, piercing, crushing, pressure, sonication, or centrifugation. In some embodiments, the seed is a dicot seed and step b) comprises (i) wounding at least part of the epicotyl and at least part of the cotyledonary node or (ii) wounding at least part of the epicotyl, at least part of the shoot apical meristem, and least part of the cotyledonary node. In some embodiments, the seed is a monocot seed and step b) comprises wounding at least part of the coleoptile, at least part of the shoot apical meristem, at least part of the leaf primordia, and at least part of the leaf axillary region. In some embodiments, the method further comprises removing a cotyledon from the explant. In some embodiments, the seed is a dicot seed and the method further comprises removing one or both cotyledons from the explant. In some embodiments, the dicot seed is a soy seed, a tobacco seed, a bean seed, a sunflower seed, a tomato seed or a pepper seed. In some embodiments, the method further comprises removing at least one primary leaf (e.g., one or two primary leaves) from the explant. In some embodiments, the method further comprises generating a plant from the wounded explant. In some embodiments, step c) comprises contacting the wounded axillary meristem region with a heterologous polynucleotide, wherein the heterologous polynucleotide comprises a selectable marker and wherein the method further comprises contacting the wounded explant or a plant or plant part generated from the wounded explant, or a combination thereof, with a selection agent to eliminate or reduce untransformed tissue. In some embodiments, the contacting with the selection agent comprises (i) adding the selection agent to a medium in which the wounded explant is maintained, (ii) adding the selection agent to a medium in which the plant is maintained, (iii) spraying the plant with the selection agent, or (iv) applying the selection agent to the wounded area of the explant or the corresponding area of the plant, or a combination thereof. In some embodiments, the contacting with the selection agent comprises (i) adding the selection agent to a medium in which the wounded explant is maintained, (ii) adding the selection agent to a medium in which the plant is maintained, and (iv) applying the selection agent to the corresponding area of the plant. In some embodiments, (i) occurs for up to 4 weeks, (ii) occurs for up to 2 weeks and (iv) occur for up to 5 weeks. In some embodiments, step (ii) is performed prior to step (iv). In some embodiments, at least part of step (ii) is performed at the same time as at least part of step (iv). In some embodiments, the selection agent is an herbicide, an antibiotic, or a non-metabolizable sugar. In some embodiments, the selection agent is glyphosate, glufosinate, spectinomycin, bensulfuron-methyl, D-xylose, mannose or kanamycin. In some embodiments, the method further comprises performing an assay on a plant generated from the wounded explant or a sample of the plant to assess for the presence or absence of transformed cells and/or to assess for the number of transformed cells. In some embodiments, the method further comprises growing the plant to produce a seed and harvesting the seed, wherein the seed optionally comprises at least part of the heterologous polynucleotide. In some embodiments, the method further comprises growing the seed to produce a progeny plant, optionally wherein the progeny plant comprises at least part of the heterologous polynucleotide. In some embodiments, the heterologous polynucleotide encodes or comprises a genome editing agent or wherein the heterologous protein comprises a genome editing agent. In some embodiments, the genome editing agent is a nuclease or a recombinase. In some embodiments, the heterologous polynucleotide comprises one or more polynucleotides encoding a Cas protein and/or a guide RNA or wherein the heterologous protein comprises a Cas protein. In some embodiments, the Cas protein is Cas9 or Cas12a, or a functional variant thereof. In some embodiments, the heterologous polynucleotide comprises an expression cassette comprising a coding sequence. In some embodiments, the expression cassette further comprises a promoter operably linked to the coding sequence. In some embodiments, the coding sequence encodes a protein or non-coding RNA of interest. In some embodiments, the contacting in step c) is performed with Agrobacterium, viral particles, microparticles, nanoparticles, cell membrane penetrating peptides, aerosol beam, chemicals, electroporation, or pressure. In some embodiments, the contacting is performed with Agrobacterium or viral particles and the contacting comprises an infection step and optionally an incubation step. In some embodiments, the infection step is performed for 30 minutes to 24 hours in darkness and the incubation step is performed for at least 2 days in darkness, optionally 4-5 days.

In other aspects, the disclosure provides an explant or plant produced by the method of any of the above-mentioned embodiments. In other aspects, the disclosure provides an explant or plant produced by a method described in the Examples. In other aspects, the disclosure provides a progeny seed produced by crossing the plant with a second plant or by self-crossing the plant. In other aspects, the disclosure provides a derivative or a commodity product produced or obtained from the plant or a part thereof.

In other aspects, the disclosure provides a method, comprising: a) providing an explant obtained from a seed, b) wounding the explant to produce a wounded explant, c) contacting the wounded explant with a heterologous polynucleotide comprising a selection marker under conditions where the heterologous polynucleotide enters the wounded explant; d) generating a plant from the wounded explant, and e) contacting the plant or a part thereof with a selection agent to eliminate or reduce untransformed tissue.

### BRIEF DESCRIPTION OF THE DRAWINGS

FIG. 1 is a diagram showing an example transformation process.

FIG. 2 is a series of drawings showing an example wounding method performed on a dicot explant removed from an imbibed seed. The embryo axis, cotyledons, and other various features of the embryo axis are shown but are not drawn to scale.

### DEFINITIONS

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

All patents, patent publications, non-patent publications referenced herein are incorporated by reference in their entireties for the teachings relevant to the sentence or paragraph in which the reference is presented. In case of a conflict in terminology, the present specification is controlling.

As used herein, the terms “a” or “an” or “the” may refer to one or more than one, unless the context clearly and unequivocally indicates otherwise. For example, “an” endogenous nucleic acid can mean one endogenous nucleic acid or a plurality of endogenous nucleic acids.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably ±0.5° C. Where the term “about” is used in the context of this invention (e.g., in combinations with temperature or molecular weight values) the exact value (i.e., without “about”) is preferred.

As used herein, an “embryo axis” comprises an epicotyl, a shoot apical meristem, a hypocotyl, a radicle, and at least one primary leaf (which may also be referred to as leaf primordia), and excludes the cotyledon(s).

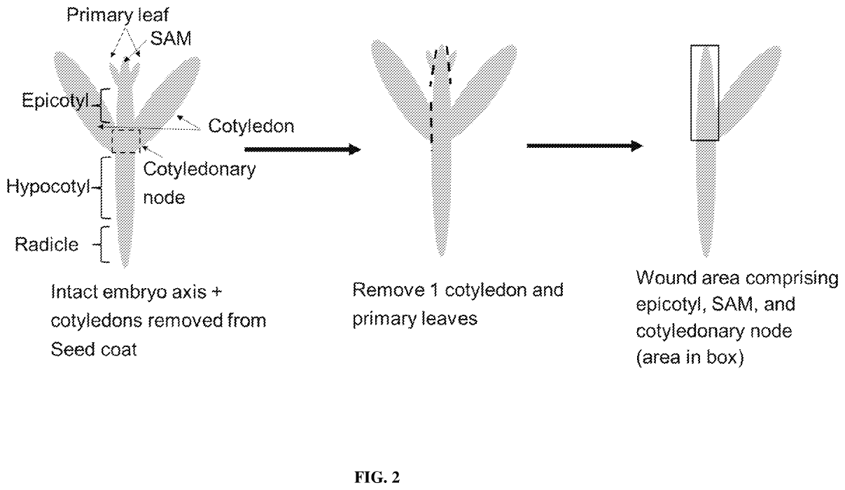

“Explant,” as used herein, refers to tissue, a piece of tissue, or pieces of tissue derived from a plant or a plant part, such as a seed. An explant can be a part of a plant, such as immature embryos, mature embryos, leaves meristems, or can be derived from a portion of the shoot, leaves, immature embryos or any other tissue of a plant or seed. An example explant relevant to the disclosure is an intact embryo axis and cotyledons removed as a single tissue from an imbibed seed (see left-most panel is FIG. 2).

As used herein, the term “expression cassette” refers to a nucleotide capable of directing expression of a particular nucleic acid sequence in a host cell. In some embodiments, the expression cassette comprises, consists essentially of or consists of one or more promoter sequences (e.g., one or more constitutive/inducible promoter sequences, one or more tissue- and/or organ-specific promoter sequences and/or one or more developmental stage-specific promoter sequences) operably linked to a nucleic acid of interest, which is operably linked to a termination sequence. Expression cassettes often comprise sequences required for proper translation of the nucleic acid sequence of interest in the host cell. The expression cassette may be chimeric in that at least one of its components is heterologous with respect to at least one of its other components. The expression cassette may be one that is naturally occurring but that has been obtained in a recombinant form useful for heterologous expression. Typically, however, the expression cassette is heterologous with respect to the host (i.e., the particular nucleic acid sequence of the expression cassette does not occur naturally in the host cell and must have been introduced into the host cell or an ancestor of the host cell by a transformation event).

As used herein, the term “genome editing agent” refers to an agent that is capable of inducing a deletion, insertion, indel, or other modification in the genome of a cell, e.g., by creating a single or double-stranded break in the genome. Examples of genome editing agents include CRISPR/Cas agents (e.g., Cas proteins and guide RNAs), transcription activator-like effector nucleases (TALENs), DNA-guided nucleases, meganucleases, recombinases, and zinc finger nucleases. Cas proteins include Cas9, Cas12a (also known as Cpf1), C2c1, C2c2, and C2c3, and functional variants thereof. Example Cas9 and Cas12a proteins include Streptococcus pyogenes Cas9 (SpCas9), Streptococcus thermophilus Cas9 (StCas9), Streptococcus pasteurianus (SpaCas9), Campylobacter jejuni Cas9 (CjCas9), Staphylococcus aureus (SaCas9), Francisella novicida Cas9 (FnCas9), Neisseria cinerea Cas9 (NcCas9), Neisseria meningitis Cas9 (NmCas9), Francisella novicida Cpf1 (FnCpf1), Acidaminococcus sp. Cpf1 (AsCpf1), or Lachnospiraceae bacterium ND2006 Cpf1 (LbCpf1). A “variant” of a Cas protein refers to a protein or polypeptide derivative of a wild type Cas protein, e.g., a protein having one or more point mutations, insertions, deletions, truncations, a fusion protein, or a combination thereof. In certain embodiments, the Cas variant is a functional variant which substantially retains the nuclease activity of or has better nuclease activity than the wild type Cas protein. Example guide RNAs include single guide RNAs and dual guide RNAs.

As used herein, the term “heterologous” refers to a polynucleotide/polypeptide at least a part of which originates from a foreign species, or, if from the same species, is substantially modified from its native form in composition and/or genomic locus by deliberate human intervention. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one nonlimiting example, a promoter may be present in a nucleic acid construct in combination with one or more regulatory element and/or coding sequences that do not naturally occur in association with that particular promoter, i.e., they are heterologous to the promoter.

As used herein, the term “in planta” when referring to a process or method step refers to a process or method step that is performed on a plant and not on excised or in vitro cultivated plant tissues or organs. For clarity, a plant includes those that have been wounded at some point during their life cycle.

The terms “nucleic acid” or “polynucleotide” are used interchangeably herein and refer to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated. The nucleic acid can be present in a vector, such as in a cell, virus or plasmid.

As used herein, the phrases “operably linked,” “operatively linked,” “operatively associated” or “in operative association” and the like, mean that elements of a nucleic acid construct such as an expression cassette or nucleic acid molecule are configured so as to perform their usual function. Thus, regulatory or control sequences (e.g., promoters) operatively associated with a nucleotide sequence are capable of effecting expression of the nucleotide sequence. For example, a promoter is operably linked with a coding sequence or functional RNA when it is capable of affecting the expression of that coding sequence or functional RNA (i.e., the coding sequence or functional RNA is under the transcriptional control of the promoter). Coding sequences in sense or antisense orientation can be operably-linked to regulatory sequences. The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

The term “plant” refers to any plant, particularly to agronomically useful plants (e.g. seed plants), and “plant cell” is a structural and physiological unit of the plant, which comprises a cell wall but may also refer to a protoplast. The plant cell may be in form of an isolated single cell or a cultured cell, or as a part of higher organized units such as for example, a plant tissue, or a plant organ differentiated into a structure that is present at any stage of a plant's development. A plant may be a monocotyledonous (monocot) or dicotyledonous (dicot) plant species.

The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like. The term “plant part” also includes explants.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

As used herein, the term “shoot apical meristem”, “shoot apex meristem” or “SAM” refers to a region of a plant containing stem cells that is located at the apex of a stem of a plant or plant seedling. In a plant seedling, the shoot apical meristem is located at the tip of a shoot.

By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear, mitochondrial and the plastid genome, and therefore includes integration of the nucleic acid into, for example, the chloroplast genome. Stable transformation as used herein can also refer to a transgene that is maintained extrachromasomally, for example, as a minichromosome.

“Selection agent” refers to an agent (e.g., a chemical) that interacts with a selectable marker to give a plant cell a selective advantage. Example selection agents are known in the art and described herein, such as glyphosate, glufosinate, spectinomycin, and kanamycin.

A “selectable marker” or “selectable marker gene” refers to a gene whose expression in a plant cell gives the cell a selective advantage. “Positive selection” refers to a transformed cell acquiring the ability to metabolize a substrate that it previously could not use or could not use efficiently, typically by being transformed with and expressing a positive selectable marker gene. This transformed cell thereby grows out of the mass of nontransformed tissue. Positive selection can be of many types from inactive forms of plant growth regulators that are then converted to active forms by the transferred enzyme to alternative carbohydrate sources that are not utilized efficiently by the nontransformed cells, for example mannose, which then become available upon transformation with an enzyme, for example phosphomannose isomerase, that allows them to be metabolized. Nontransformed cells either grow slowly in comparison to transformed cells or not at all. Other types of selection may be due to the cells transformed with the selectable marker gene gaining the ability to grow in presence of a negative selection agent, such as an antibiotic or an herbicide, compared to the ability to grow of non-transformed cells. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

The term “transformation” as used herein refers to the transfer of a nucleic acid into a host cell, which includes integration into a chromosome, heritable extrachromosomal events and transient transfer. In some particular embodiments, the introduction into a plant, plant part and/or plant cell is via bacterial-mediated transformation, particle bombardment transformation (also called biolistic particle transformation), calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of a nucleic acid into the plant, plant part and/or cell thereof, or a combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (2002, Cell Mol Biol Lett 7:849-858 (2002)).

As used herein, the terms “transformed” and “transgenic” refer to any plant, plant cell, callus, plant tissue, or plant part that contains all or part of at least one heterologous polynucleotide. In some embodiments, all or part of the heterologous polynucleotide is stably integrated into a chromosome or stable extra-chromosomal element, so that it is passed on to successive generations.

### DETAILED DESCRIPTION

Provided herein are methods for transformation of plants, and related compositions.

In some embodiments, the disclosure provides a method comprising (a) providing an explant of a seed, wherein the explant comprises an embryo axis and a cotyledon, (b) wounding at least part of a region of the explant to produce a wounded explant, and (c) contacting the wounded explant with a heterologous polynucleotide and/or heterologous protein under conditions where the heterologous polynucleotide and/or heterologous protein enters the wounded explant. In some embodiments, if the seed is a dicot seed then the region of the explant comprises an epicotyl, a shoot apical meristem, and cotyledonary node. In some embodiments, if the seed is a monocot seed then the region of the explant comprises a coleoptile, a shoot apical meristem, a leaf primordia, and a leaf axillary region.

In some embodiments, the seed is an imbibed seed. In some embodiments, the seed is a mature seed, e.g., a mature imbibed seed. In some embodiments, the seed is a mature sterilized seed, e.g., a mature sterilized imbibed seed. In some embodiments, the seed is a dicot seed, e.g., a soy seed, a tobacco seed, a bean seed, a sunflower seed, a tomato seed or a pepper seed. In some embodiments, the seed is a monocot seed, e.g., a maize (corn) seed, a barley seed, an oat seed, a rice seed, a sorghum seed, a sugarcane seed or a wheat seed. In some embodiments, the seed has been imbibed in a liquid medium or incubated on a solid medium for up to 48 hours (e.g., between 4-48 hours, 4-24 hours, or 12-18 hours). In some embodiments, the liquid or solid medium comprises Gamborg's B5 basal medium with or without sucrose, and optionally a cytokinin such as zeatin or BAP.

In some embodiments, the wounded explant is produced by removing a seed coat from the seed (e.g., a dicot seed) to release the explant and performing the steps of the method above on the explant. In some embodiments, the explant is produced by providing a seed (e.g., a monocot seed) that has an embryo axis and cotyledon emerging or emerged from it and performing the steps of the method above on the emerging or emerged embryo axis and cotyledon.

In some embodiments, the disclosure provides method of producing a chimeric plant with at least one transgenic shoot, comprising: (a) providing a plant comprising an axillary meristem and a shoot apical meristem, (b) removing or wounding at least part of the axillary meristem to produce a wounded axillary meristem region, (c) contacting the wounded axillary meristem region with a heterologous polynucleotide and/or heterologous protein under conditions where the heterologous polynucleotide and/or heterologous protein enters wounded axillary meristem region, (d) Removing the shoot apical meristem or suppressing the growth of the shoot apical meristem at the same time as step b) or step c) or after step c) to produce a wounded explant, (e) Culturing the wounded explant in a medium in vitro to promote cell proliferation and regeneration, and (f) Growing the wounded explant in a proper medium and applying selection agent to the resulting plant in planta to select for a transgenic shoot.

In some embodiments, step c) comprises contacting the wounded axillary meristem region with a heterologous polynucleotide, wherein the heterologous polynucleotide comprises a selectable marker and wherein the method further comprises contacting the wounded explant or a plant or plant part generated from the wounded explant, or a combination thereof, with a selection agent to eliminate or reduce untransformed tissue.

In some embodiments, the contacting with the selection agent comprises adding the selection agent to a medium in which the wounded explant is maintained.

In some embodiments, the applying selection agent to the resulting plant in planta comprises (i) adding the selection agent to a medium in which the plant is maintained, (ii) spraying the plant with the selection agent, or (iii) applying the selection agent to the wounded area of the explant or the corresponding area of the plant, or a combination thereof.

In some embodiments, applying selection agent to the plant in planta comprises (i) adding the selection agent to a medium in which the plant is maintained, (ii) spraying the plant with the selection agent, or (iii) applying the selection agent to the wounded area of the explant or the corresponding area of the plant, optionally wherein (i) occurs for up to 4 weeks, (ii) occurs for up to 2 weeks and (iii) occur for up to 5 weeks.

In some embodiments, step (i) is performed prior to step (iii).

In some embodiments, at least part of step (i) is performed at the same time as at least part of step (iii).

In some embodiments, the wounding is performed by a method comprising cutting (e.g., with a scalpel or other bladed instrument), piercing (e.g., with a needle or other pointed instrument), crushing (e.g., with the flat side of a scalpel blade or other appropriate instrument), pressure (e.g., vacuum), sonication, or centrifugation (e.g., with particles).

In some embodiments, the seed is a dicot seed and the wounding at least part of the region of the embryo axis comprises wounding at least part of the epicotyl, at least part of the shoot apical meristem, or least part of the cotyledonary node, or a combination thereof. In some embodiments, wounding at least part of the region of the embryo axis comprises (i) wounding at least part of the epicotyl and at least part of the cotyledonary node or (ii) wounding at least part of the epicotyl, at least part of the shoot apical meristem, and least part of the cotyledonary node.

In some embodiments, the seed is a monocot seed and the wounding at least part of the region of the embryo axis comprises wounding at least part of the coleoptile, at least part of the shoot apical meristem, at least part of the leaf primordia, at least part of the leaf axillary region, or a combination thereof. In some embodiments, wounding at least part of the region of the embryo axis comprises wounding at least part of the coleoptile, at least part of the shoot apical meristem, at least part of the leaf primordia, and at least part of the leaf axillary region.

In some embodiments of the method, the method further comprises removing a cotyledon from the explant. In some embodiments, the seed is a monocot seed and the method further comprises removing one cotyledon from the explant. In some embodiments, the monocot seed, is a maize (corn) seed, a barley seed, an oat seed, a rice seed, a sorghum seed, a sugarcane seed or a wheat seed. In some embodiments, the seed is a dicot seed and the method further comprises removing one or both cotyledons from the explant. In some embodiments, the dicot seed is a soy seed, a tobacco seed, a bean seed, a sunflower seed, a tomato seed or a pepper seed.

In some embodiments of the method, the method further comprises removing at least one primary leaf from the explant. In some embodiments of the method, the method further comprises removing one or both of the primary leaves from the explant. In some embodiments, the removing is done by cutting off the primary leaves from the explant.

In some embodiments of the method, the method further comprises generating a plant from the wounded explant. In some embodiments, the generating comprises recovering the wounded explant in a recovery medium, e.g., for up to 4 weeks. In some embodiments, the selection medium comprises Gamborg's B5 basal medium, MS iron, Gamborg's B5 vitamins, MES, glutamine, asparagine, timentin and cytokinin (e.g., BAP or zeatin riboside).

In some embodiments of the method, the heterologous polynucleotide comprises a selectable marker and the method further comprises contacting the wounded explant or a plant or plant part generated from the wounded explant, or a combination thereof, with a selection agent to eliminate or reduce untransformed tissue. In some embodiments, the selection agent is an herbicide, an antibiotic, or a non-metabolizable sugar. In some embodiments, the selection agent is glyphosate, glufosinate, mesotrione, isoxaflutole, bicyclopyrone, tembotrione, butafenacil, spectinomycin, bensulfuron-methyl, imazapyr, dicamba, 2,4-D, Haloxyfop, Fluazifop, D-xylose, mannose or kanamycin.

In some embodiments, the selection process comprises using one or more selection steps, selectable markers (e.g., EPSPS or ALS) and/or selection agents (e.g., glyphosate or bensulfuron-methyl) described in the Examples.

Examples of selectable markers include, but are not limited to, genes that provide resistance or tolerance to antibiotics such as kanamycin (Dekeyser et al. 1989, Plant Phys 90: 217-23), spectinomycin (Svab and Maliga 1993, Plant Mol Biol 14: 197-205), streptomycin (Maliga et al. 1988, Mol Gen Genet 214: 456-459), hygromycin B (Waldron et al. 1985, Plant Mol Biol 5: 103-108), bleomycin (Hille et al. 1986, Plant Mol Biol 7: 171-176), sulphonamides (Guerineau et al. 1990, Plant Mol Biol 15: 127-136), streptothricin (Jelenska et al. 2000, Plant Cell Rep 19: 298-303), or chloramphenicol (De Block et al. 1984, EMBO J 3: 1681-1689). Other selectable markers include genes that provide resistance or tolerance to herbicides, such as the S4 and/or Hra mutations of acetolactate synthase (ALS) that confer resistance to herbicides including sulfonylureas, imidazolinones, triazolopyrimidines, and pyrimidinyl thiobenzoates; 5-enol-pyrovyl-shikimate-3-phosphate-synthase (EPSPS) genes, including but not limited to those described in U.S. Pat. Nos. 4,940,935, 5,188,642, 5,633,435, 6,566,587, 7,674,598 (as well as all related applications) and the glyphosate N-acetyltransferase (GAT) which confers resistance to glyphosate (Castle et al. 2004, Science 304:1151-1154, and U.S. Patent Application Publication Nos. 20070004912, 20050246798, and 20050060767); BAR which confers resistance to glufosinate (see e.g., U.S. Pat. No. 5,561,236); aryloxy alkanoate dioxygenase or AAD-1, AAD-12, or AAD-13 which confer resistance to 2,4-D; genes such as Pseudomonas HPPD which confer HPPD resistance; Sprotophorphyrinogen oxidase (PPO) mutants and variants, which confer resistance to peroxidizing herbicides including fomesafen, acifluorfen-sodium, oxyfluorfen, lactofen, fluthiacet-methyl, saflufenacil, flumioxazin, flumiclorac-pentyl, carfentrazone-ethyl, sulfentrazone); and genes conferring resistance to dicamba, such as dicamba monoxygenase (Herman et al. 2005, J Biol Chem 280: 24759-24767 and U.S. Pat. No. 7,812,224 and related applications and patents). Other examples of selectable markers can be found in Sundar and Sakthivel (2008, J Plant Physiology 165: 1698-1716), herein incorporated by reference. Additional selectable markers for use in the disclosure are known in the art such as Phosphinothricin N-acetyl transferase (PAT) and Aminoglycoside 3′-adenylyiltransferase (aadA) (see, e.g., Rosellini (2012) Selectable Markers and Reporter Genes: A Well Furnished Toolbox for Plant Science and Genetic Engineering, Critical Reviews in Plant Sciences, 31:5, 401-453).

Other selection systems include using drugs, metabolite analogs, metabolic intermediates, and enzymes for positive selection or conditional positive selection of transgenic plants. Examples include, but are not limited to, a gene encoding phosphomannose isomerase (PMI) where mannose is the selection agent, or a gene encoding xylose isomerase where D-xylose is the selection agent (Haldrup et al. 1998, Plant Mol Biol 37: 287-96). Finally, other selection systems may use hormone-free medium as the selection agent. One non-limiting example the maize homeobox gene kn1, whose ectopic expression results in a 3-fold increase in transformation efficiency (Luo et al. 2006, Plant Cell Rep 25: 403-409). Examples of various selectable markers and genes encoding them are disclosed in Miki and McHugh (J Biotechnol, 2004, 107: 193-232; incorporated by reference).

In some embodiments of the disclosure, the selectable marker may be plant derived. An example of a selectable marker which can be plant derived includes, but is not limited to, 5-enolpyruvylshikimate-3-phosphate synthase (EPSPS). The enzyme 5-enolpyruvylshikimate-3-phosphate synthase (EPSPS) catalyzes an essential step in the shikimate pathway common to aromatic amino acid biosynthesis in plants. The herbicide glyphosate inhibits EPSPS, thereby killing the plant. Transgenic glyphosate-tolerant plants can be created by the introduction of a modified EPSPS transgene which is not affected by glyphosate (for example, U.S. Pat. No. 6,040,497; incorporated by reference). Other examples of a modified plant EPSPS which can be used as a selectable marker in the presence of glyphosate includes a P106L mutant of rice EPSPS (Zhou et al 2006, Plant Physiol 140: 184-195) and a P106S mutation in goosegrass EPSPS (Baerson et al 2002, Plant Physiol 129: 1265-1275). Other sources of EPSPS which are not plant derived and can be used to confer glyphosate tolerance include but are not limited to an EPSPS P101S mutant from Salmonella typhimurium (Comai et al 1985, Nature 317: 741-744) and a mutated version of CP4 EPSPS from Agrobacterium sp. Strain CP4 (Funke et al 2006, PNAS 103: 13010-13015). Although the plant EPSPS gene is nuclear, the mature enzyme is localized in the chloroplast (Mousdale and Coggins 1985, Planta 163:241-249). EPSPS is synthesized as a preprotein containing a transit peptide, and the precursor is then transported into the chloroplast stroma and proteolytically processed to yield the mature enzyme (della-Cioppa et al. 1986, PNAS 83: 6873-6877). Therefore, to create a transgenic plant which has tolerance to glyphosate, a suitably mutated version of EPSPS which correctly translocates to the chloroplast could be introduced. Such a transgenic plant then has a native, genomic EPSPS gene as well as the mutated EPSPS transgene. Glyphosate could then be used as a selection agent during the transformation and regeneration process, whereby only those plants or plant tissue that are successfully transformed with the mutated EPSPS transgene survive.

In some embodiments of the method, the contacting with the selection agent comprises adding the selection agent to a medium (e.g., soil or hydroponics) in which the plant is growing (e.g., by watering or applying to the soil or other medium a composition comprising the selection agent, such as between 1 uM to 1M of a selection agent, e.g., 10 uM to 500 uM of glyphosate or 0.1 uM to 10 uM Bensulfuron-methyl), spraying the plant with the selection agent (e.g., with a sprayable composition comprising the selection agent, such as 1 uM to 1M of a selection agent, e.g., between 10 uM to 50 mM glyphosate or 0.1 uM to 10 uM Bensulfuron-methyl), or applying the selection agent (such as between 1 uM to 1M of a selection agent, e.g., 10 uM to 200 uM glyphosate or 0.1 uM to 10 uM Bensulfuron-methyl) to the area of the plant corresponding to the wounded area of the explant (e.g., using a solution, gel, absorbable material (e.g., cotton ball) or other material that can release the selection agent (such as onto the area of the plant corresponding to the wounded area of the explant). In some embodiments, the contacting with the selection agent occurs for at least one day, at least one week, at least two weeks, at least three weeks, at least four weeks, at least five weeks, or longer. In some embodiments, the contacting with the selection agent occurs for between 1-11, 1-10, 1-9, 1-8, 1-7, 1-6, 1-5, 1-4, 1-3, 1-2, 2-11, 2-10, 2-9, 2-8, 2-7, 2-6, 2-5, 2-4, 2-3, 3-11, 3-10, 3-9, 3-8, 3-7, 3-6, 3-5, 3-4, 4-11, 4-10, 4-9, 4-8, 4-7, 4-6, 4-5, 5-11, 5-10, 5-9, 5-8, 5-7, or 5-6 weeks. In some embodiments, the contacting with the selection agent occurs for between 1 day to 6 weeks. In some embodiments, at least one step of the contacting with the selection agent occurs in planta.

In some embodiments, the contacting with the selection agent comprises (i) adding the selection agent to a medium in which the wounded explant is maintained, (ii) adding the selection agent to a medium in which the plant is maintained (e.g., soil or hydroponics), (iii) spraying the plant with the selection agent, or (iv) applying the selection agent to the wounded area of the explant or the corresponding area of the plant, or a combination thereof (e.g., i and ii; i, ii, and iii; i, ii, and iv; i, ii, iii and iv; i, iii and iv; i and iii; i and iv; ii and iii; ii and iv; etc.). In some embodiments, the contacting with the selection agent comprises (i) adding the selection agent to a medium in which the wounded explant is maintained, (ii) adding the selection agent to a medium in which the plant is maintained (e.g., soil or hydroponics), and (iv) applying the selection agent to the corresponding area of the plant. In some embodiments, (i) occurs for up to 4 weeks (e.g., between 1 and 14, 1 and 10, 1 and 2, or 2 and 7 days), (ii) occurs for up to 2 weeks and (iv) occur for up to 5 weeks. In some embodiments, step (ii) is performed prior to step (iv). In some embodiments, at least part of step (ii) is performed at the same time as at least part of step (iv), e.g., steps (ii) and (iv) overlap for at least 1, 2, 3, 4, 5, 6, 7 or more days.

In some embodiments of the method, the method further comprises performing an assay on a plant generated from the wounded explant or a sample of the plant to assess for the presence or absence of transformed cells and/or to assess for the number of transformed cells. Example assays include fluorescent protein detection, qPCR, real-time PCR, immunoassays, and the like.

In some embodiments of the method, the method further comprises growing the plant to produce a seed (e.g., one seed, two seeds, ten seeds, twenty seeds, fifty seeds or more) optionally comprising at least part of the heterologous polynucleotide and harvesting the seed. In some embodiments, all seeds produced by the plant comprise at least part of the heterologous polynucleotide. In some embodiments, at least one seed, or more seeds (e.g., at least 10%, at least 20%, at least 30%, at least 40%, at least 50%, at least 60%, at least 70%, at least 80%, or at least 90%) of the seeds, produced by the plant comprise at least part of the heterologous polynucleotide. In some embodiments of the method, the method further comprises growing the seed(s) to produce a progeny plant(s), optionally comprising at least part of the heterologous polynucleotide.

In some embodiments of the method, the heterologous polynucleotide encodes a genome editing agent, e.g., a CRISPR/Cas agent, a TALEN, a DNA-guided nuclease, a meganuclease, a recombinase, or a zinc finger nuclease. In some embodiments of the method, the heterologous protein comprises a genome editing agent, e.g., a Cas protein, a TALEN, a DNA-guided nuclease, a meganuclease, a recombinase, or a zinc finger nuclease. In some embodiments, the heterologous polynucleotide comprises one or more polynucleotides encoding a Cas protein and/or a guide RNA. In some embodiments, the heterologous polynucleotide comprises one or more guide RNAs, optionally wherein the heterologous polynucleotide is comprised within a ribonucleoprotein (RNP) with a Cas protein. In some embodiments, the Cas protein is Cas9 or Cas12a, or a functional variant thereof.

In some embodiments of the method, the heterologous polynucleotide comprises an expression cassette comprising a coding sequence. In some embodiments of the method, the coding sequence encodes a protein or non-coding RNA of interest. In some embodiments, the protein or non-coding RNA of interest confers one or more desired traits on a plant, such as enhanced growth, enhanced yield, drought tolerance, salt tolerance, herbicide tolerance, insect resistance, pest resistance, disease resistance, temperature tolerance, enhanced nitrogen utilization and the like. In some embodiments, the coding sequence encodes a genome editing agent, such as a Cas protein and/or a guide RNA. In some embodiments, the heterologous polynucleotide comprises a coding sequence encoding a protein or non-coding RNA of interest and a coding sequence a selection marker. In some embodiments of the method, the expression cassette further comprises a promoter operably linked to the coding sequence(s). The promoter may be, e.g., a constitutive promoter, a tissue-specific promoter, or an inducible promoter.

In some embodiments of the method, the contacting in step c) is performed with Agrobacterium, viral particles, microparticles or nanoparticles (e.g., gold or tungsten microparticles or nanoparticles), cell membrane penetrating peptides, aerosol beam, chemicals, electroporation, or pressure (e.g., vacuum). In some embodiments, the contacting in step (d) is performed with Agrobacterium. In some embodiments, the contacting in step (c) is performed with viral particles. In some embodiments, the contacting in step (c) is performed with gold or tungsten particles, such as microparticles or nanoparticles. In some embodiments, the contacting in step (c) is performed with cell membrane penetrating peptides. In some embodiments, the contacting in step (c) is performed with an aerosol beam. In some embodiments, the contacting in step (c) is performed with chemicals. In some embodiments, the contacting in step (c) is performed with electroporation. In some embodiments, the contacting in step (c) is performed with pressure (e.g., vacuum).

In some embodiments of the method, the contacting is performed with Agrobacterium or viral particles and the contacting comprises an infection step and an incubation step. In some embodiments of the method, the infection step is performed in darkness or in light or in a light/dark cycle for at least 30 minutes, e.g., 30 minutes to 24 hours, such as 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 hours, and the incubation step is performed in darkness or in light or in a light/dark cycle for at least 1 day, at least 2 days, at least 3 days, at least 4 days, at least 5 days, at least 6 days, at least 7 days or more, e.g., 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 days. In some embodiments, the infection step comprises contacting the wounded explant with a solution, gel, absorbable material or other material that contains the Agrobacterium or viral particles. In some embodiments, after incubation, antibiotics (e.g., Timentin, Cefotaxime and/or Vancomycin) are applied to eliminate the Agrobacterium or viral particles.

Agrobacterium-mediated transformation is a commonly used method for transforming plants because of its relatively high efficiency and increased throughput of transformation and because of its broad utility with many different species. Agrobacterium-mediated transformation typically involves transfer of a binary vector carrying the foreign DNA of interest to an appropriate Agrobacterium strain that may depend on the complement of vir genes carried by the host Agrobacterium strain either on a co-resident Ti plasmid or chromosomally (see, e.g., Uknes et al 1993, Plant Cell 5:159-169). The transfer of the recombinant binary vector to Agrobacterium can be accomplished, e.g., by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (see, e.g., Höfgen and Willmitzer 1988, Nucleic Acids Res 16:9877). Transformation of a plant by recombinant Agrobacterium usually involves incubation of the Agrobacterium with explants from the plant. Transformed tissue is typically regenerated in the presence of a selection agent for a selectable marker that is located between the binary plasmid T-DNA borders.

In other aspects, the disclosure provides a method comprising wounding an explant obtained from a seed (e.g., an imbibed or germinated seed), transforming the wounded explant, generating a plant from the wounded explant, contacting the wounded explant with a heterologous polynucleotide comprising a selection marker under conditions where the heterologous polynucleotide enters the wounded explant, and performing at least one selection step in planta. In some embodiments, the wounded explant is an explant as described above or in the Examples. In some embodiments, the wounded explant is an embryo axis with the cotyledons removed (see, e.g., the explants described in U.S. Pat. No. 7,001,754). In some embodiments, the wounded explant is an embryo axis wherein one cotyledon and the radicle has been removed (see, e.g., the explants described in US Patent Application Publication No. US2004034889). In some embodiments, the wounded explant is a half of a seed (see, e.g., the explants described in U.S. Pat. No. 7,473,822).

In some embodiments, the in planta selection step(s) comprises using one or more selection steps, selectable markers (e.g., EPSPS or ALS) and/or selection agents (e.g., glyphosate or bensulfuron-methyl) described in the Examples. In some embodiments of the method, the in planta selection step(s) comprises adding the selection agent to a medium (e.g., soil or hydroponics) in which the plant is growing (e.g., by watering or applying to the soil or other medium a composition comprising the selection agent), spraying the plant with the selection agent (e.g., with a sprayable composition comprising the selection agent), or applying the selection agent to the area of the plant corresponding to the wounded area of the explant (e.g., using a solution, gel, absorbable material (e.g., cotton ball) or other material that can release the selection agent (such as onto the area of the plant corresponding to the wounded area of the explant), or a combination thereof. In some embodiments, the in planta selection step occurs for at least one day, at least one week, at least two weeks, at least three weeks, at least four weeks, at least five weeks, or longer. In some embodiments, the contacting with the selection agent occurs for between 1-11, 1-10, 1-9, 1-8, 1-7, 1-6, 1-5, 1-4, 1-3, 1-2, 2-11, 2-10, 2-9, 2-8, 2-7, 2-6, 2-5, 2-4, 2-3, 3-11, 3-10, 3-9, 3-8, 3-7, 3-6, 3-5, 3-4, 4-11, 4-10, 4-9, 4-8, 4-7, 4-6, 4-5, 5-11, 5-10, 5-9, 5-8, 5-7, or 5-6 weeks.

Other aspects of the disclosure relate to a method of transformation comprising any one or more steps of a method described in the Examples. Other aspects of the disclosure relate to an explant, plant or plant part produced by any of the methods described above or elsewhere herein, including in the Examples. Other aspects of the disclosure relate to progeny seed produced by crossing the plant produced by any of the methods described above or elsewhere herein with a second plant or by selfing the plant. Other aspects of the disclosure relate to a derivative or a commodity product produced or obtained from the plant or plant part produced by any of the methods described above or elsewhere herein. In some embodiments, the commodity product is selected from the group consisting of whole or processed seeds, flour, protein isolates, concentrates, liquids, syrups, pastes, sauces or other food or product produced from the plant or plant part.

In some embodiments, the disclosure provides a method comprising (a) providing an explant of a seed, wherein the explant comprises an embryo axis and a cotyledon, and (b) contacting the explant with a heterologous polynucleotide and/or heterologous protein under conditions where the heterologous polynucleotide and/or heterologous protein enters the explant. In some embodiments, if the seed is a dicot seed then the region of the explant comprises an epicotyl, a shoot apical meristem, and cotyledonary node. In some embodiments, if the seed is a monocot seed then the region of the explant comprises a coleoptile, a shoot apical meristem, a leaf primordia, and a leaf axillary region.

In some embodiments, the disclosure provides a method comprising (a) providing an explant of a seed, wherein the explant comprises an embryo axis and a cotyledon, and (b) contacting the explant with a heterologous polynucleotide and/or heterologous protein under conditions where the heterologous polynucleotide and/or heterologous protein enters the explant. In some embodiments, if the seed is a dicot seed then the region of the explant comprises an epicotyl, a shoot apical meristem, and cotyledonary node. In some embodiments, if the seed is a monocot seed then the region of the explant comprises a coleoptile, a shoot apical meristem, a leaf primordia, and a leaf axillary region. In some embodiments, the contacting in step (b) is performed with gold or tungsten particles, such as microparticles or nanoparticles. In some embodiments of the method, the heterologous polynucleotide encodes a genome editing agent, e.g., a CRISPR/Cas agent, a TALEN, a DNA-guided nuclease, a meganuclease, a recombinase, or a zinc finger nuclease. In some embodiments of the method, the heterologous protein comprises a genome editing agent, e.g., a Cas protein, a TALEN, a DNA-guided nuclease, a meganuclease, a recombinase, or a zinc finger nuclease. In some embodiments, the heterologous polynucleotide comprises one or more polynucleotides encoding a Cas protein and/or a guide RNA. In some embodiments, the heterologous polynucleotide comprises one or more guide RNAs, optionally wherein the heterologous polynucleotide is comprised within a ribonucleoprotein (RNP) with a Cas protein. In some embodiments, the Cas protein is Cas9 or Cas12a, or a functional variant thereof.

In some embodiments of the method, the heterologous polynucleotide comprises an expression cassette comprising a coding sequence. In some embodiments of the method, the coding sequence encodes a protein or non-coding RNA of interest. In some embodiments, the protein or non-coding RNA of interest confers one or more desired traits on a plant, such as enhanced growth, enhanced yield, drought tolerance, salt tolerance, herbicide tolerance, insect resistance, pest resistance, disease resistance, temperature tolerance, enhanced nitrogen utilization and the like. In some embodiments, the coding sequence encodes a genome editing agent, such as a Cas protein and/or a guide RNA. In some embodiments, the heterologous polynucleotide comprises a coding sequence encoding a protein or non-coding RNA of interest and a coding sequence a selection marker. In some embodiments of the method, the expression cassette further comprises a promoter operably linked to the coding sequence(s). The promoter may be, e.g., a constitutive promoter, a tissue-specific promoter, or an inducible promoter.

Hereinafter, the present invention will be described in detail by the following examples. However, the following examples are illustrative of the present invention, and the scope of the present invention is not limited by the following examples.

### EXAMPLES

### Example 1: The Process Flow of the Novel Semi-In-Planta Transformation Method

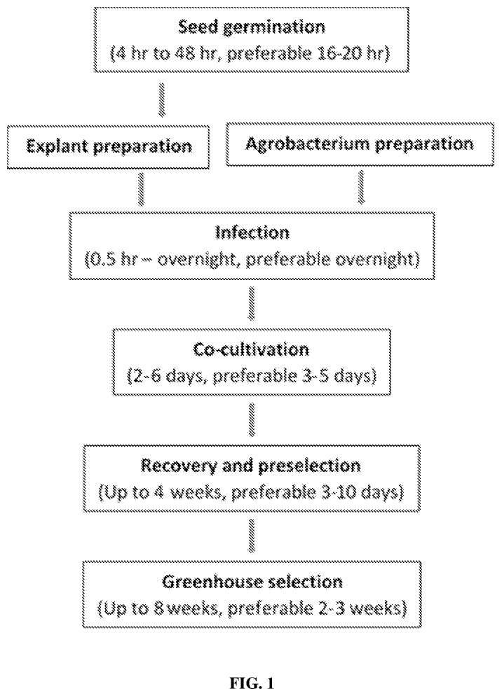

General semi-in-planta transformation protocol that is applicable to wide range of crops and their varieties are described below and shown in FIG. 1.

### 1. Target Tissue Preparation:

Sterilized or unsterilized mature or immature seeds, preferably sterilized mature seeds, are used. Prior to Agrobacterium inoculation, soak seeds in water or other liquid medium for 4-48 hours, preferably overnight. Alternatively, sterilized seeds can also be germinated in solid medium overnight. The seeds are incubated at 22-24° C. for at least 16-20 hours.

The seed coat is removed from the soaked or germinated seed. The seed is then wounded by carefully cutting into region comprising the epicotyl and shoot apical meristem (e.g., with a scalpel blade) without completely detaching the hypocotyl from cotyledons. A preferred alternative method is to carefully remove and discard one of the cotyledons, and then wound the region comprising the epicotyl, apical meristem and cotyledonary node (e.g., with the sharp end of a scalpel blade). As another alternative, the primary leaves and one of the cotyledons can be removed before wounding the region.

### 2. Agrobacterium Suspension Preparation:

Different Agrobacterium tumefaciens strain can be used for transformation, preferably such as EHA101 or Chry5 (various versions of each strain may be used, including recA−). Agrobacterium harbors binary vectors containing selectable marker and gene(s) of interest. Example constructs contain EPSPS or Acetolactate synthase (ALS) selectable markers. Agrobacterium culture is streaked from −80° C. from glycerol stock onto plates containing appropriate antibiotics and grown in a 22-28° C. incubator, preferably, 23° C. Before inoculation with explants, Agrobacterium cells are collected from the plate, uniformly suspended in liquid infection medium in a sterile disposable 50 ml centrifuge tube, and diluted to OD A660 of approximately 0.20 to 1.0, preferably an OD of approximately 0.3 to 0.6. Acetosyringone is added to induce virulence gene expression. Preferably, dithiothreitol (DTT) is added.

### 3. Infection and Incubation

The wounded explants are immediately infected with Agrobacterium by immersing them with the Agrobacterium suspension, and then incubated for at least 30 minutes or up to overnight in dark at room temperature. Alternatively, the infection of explants can also be carried out in the presence of Agrobacterium suspension, e.g., by adding the Agrobacterium suspension on the wounding region first, and then wounding the explants; or by dipping a scalpel blade in Agrobacterium suspension and then using the blade to wound the explants, or by directly wounding the explants in Agrobacterium suspension. After infection, the explants are removed from the Agrobacterium suspension and transferred to incubation on a petri dish in an airtight plastic container or solid medium without using DTT. The incubation plates are incubated for 3 to 6 days (preferably 4-5 days) at 21-23° C. in the dark, with explants placed adaxial side up.

### 4. Recovery and Optional Pre-Selection In Vitro

After incubation, liquid medium preferably, with or without corresponding selection agent, is added to just immerse the explants in incubation plates. Alternatively, the explants can be cultured on solid or semi-solid medium with or without corresponding selection agent. The cultures are then incubated at 23-28° C., preferable 25° C. under light for up to 4 weeks before being transplanted to soil for further selection and transgenic shoot regeneration and recovery. Selection agent for EPSPS gene is 25-500 μM glyphosate, preferably 100 μM. Selection agent for ALS gene is 0.01-1 mM Bensulfuron-methyl, preferably 1-3 μM. Other corresponding selection agents can also be used.

### 5. Selection and Recovery of Transgenic Plants

After recovery and optional pre-selection, the infected explants are transplanted to soil for further selection and development of transgenic shoots. The plants are placed in a growth chamber under 16 hour light/8 hour dark conditions in trays. A small cotton ball soaked in selection solution is mounted on the infected area (“top selection”) of each plant. The trays are covered with a dome to maintain high humidity. The cotton balls are changed 1-2 times weekly. Top selection can be performed for up to 2 weeks. After one week top selection, selection solution can be watered into soil pots (“bottom selection”). Alternatively, just bottom or just top selection may be used, or just spaying the selection solution with corresponding selection agent on the explants for 1-5 weeks The selection watering was done once a week, for 3-5 weeks.

### 6. Transgenic Plant Identification and Confirmation

Transgenic shoots develop without apparent abnormal phenotype and can be distinguished from chimeras which are expected to have yellow and green leaves in developed shoots or a chimeric sector in a transgenic leaf. The true transgenic shoots can be confirmed by molecular analysis such as Taqman analysis. Two to three leaves are sampled from different sets of trifoliate in one transgenic shoot and subjected to analysis the presence of selectable marker, binary vector backbone and gene-of-interest.

### 7. Inheritance Analysis of Transgene

Seeds harvested from independent transgenic shoots are germinated and leaves are sampled for molecular analysis such as Taqman analysis for the presence of selectable marker, binary vector backbone and gene-of-interest.

### Example 2: Soybean Transformation

### Detailed Description of the Semi-In-Planta Soybean Transformation Method

### 1. Target Tissue Preparation:

Sterilized mature soybean (Glycine max) seeds were used. Prior to Agrobacterium inoculation, seeds were soaked in sterilized H2O or liquid medium Soy1 for overnight (12-18 hours) at 22-24° C. Liquid medium Soy 1 contains 3.1 g/L Gamborg's B5 basal medium and 2 mg/L BAP

The seed coat was removed from the soaked seed. One of the cotyledons and both primary leaves were carefully removed and discarded. Next, the region containing the epicotyl, apical meristem and the cotyledonary node were wounded by making several cuts with the sharp end of scalpel blade. The wounding method is shown in FIG. 2.

### 2. Agrobacterium Suspension Preparation:

Agrobacterium tumefaciens strain [Chry5d3 recA−] was used. The Agrobacterium harbored a binary vector containing a selectable marker and gene of interest, specifically construct 23093 containing EPSPS selectable marker driven by translation elongation factor EF-1 alpha/Tu promoter and AmCyan gene driven by Cestrum Yellow leaf curl virus promoter (prCMP), construct18891 containing codon-optimized Acetolactate synthase (ALS) double mutant (P191A, W568L) from Nicotiana tabacum for soybean and AmCyan gene driven by prCMP, and construct 22296 containing codon-optimized Acetolactate synthase (ALS) double mutant (P191A, W568L) from Nicotiana tabacum for soybean driven by translation elongation factor EF-1 alpha/Tu promoter, including the first intron and neighboring 5′-UTR, from soybean (Williams 82) prGmEF-02. Agrobacterium culture was streaked from −80° C. from glycerol stock onto YP plates containing 100 mg/L ampicillin and 500 mg/L spectinomycin appropriate antibiotics and grown in a 23° C. incubator. Before inoculation with explants, Agrobacterium cells were collected from the plate and were uniformly suspended in liquid infection medium SoyInf in a sterile disposable 50 ml centrifuge tube and diluted to OD A660 of approximately 0.3 to 0.6. A final concentration of 40-80 mg/L (200-400 uM) acetosyringone was added to induce virulence gene expression. Dithiothreitol (DTT) was added to a final concentration of 150 μg/ml. SoyInf contains 1.1 g/L MS basal salt mixture, 20 g/L sucrose, 10 g/L glucose, 4 g/L MES, 1 ml/L Gamborg's B5 vitamins (1000×) and 2 mg/L zeatin riboside.

### 3. Infection and Co-Cultivation

The wounded explants were immediately infected with Agrobacterium by immersing them with the Agrobacterium suspension, and then incubated for 18 hours in dark at room temperature. After infection, the explants were removed from the Agrobacterium suspension and transferred to petri dishes for co-cultivation in an airtight plastic container. The co-cultivation plates were incubated for 3-5 days at 22±1° C. in the dark, with explants placed adaxial side up.

### 4. Recovery and Pre-Selection In Vitro

After co-cultivation, liquid medium Soy 2, with or without corresponding selection agent (glyphosate or bensulfuron-methyl), was added to just immerse the explants in co-cultivation plates. The cultures were ten incubated at 25° C. under light for up to 4 weeks before transplanted to soil for further selection and transgenic shoot regeneration and recovery. The details of the pre-selection steps for different experimental conditions tested are shown in Table 1 and 2. Soy2 contains 3.1 g/L Gamborg's B5 basal medium, 5 ml MS iron (200×), 1 ml/L Gamborg's B5 vitamins (1000×), 1 g/L MES, 100 mg/L glutamine, 100 mg/L asparagine, 300 mg/L timentin and 2 mg/L BAP. Selection agent for EPSPS gene was 100-300 μM glyphosate. Selection agent for ALS gene was 1 to 5 μM bensulfuron-methyl.

### 5. Selection and Recovery of Transgenic Plants

After pre-selection, the infected explants were transplanted to soil for further selection and development of transgenic shoots.

### 1). Glyphosate Selection to Generate Transgenic Events:

Selection for event regeneration: the plants were placed in a growth chamber under 16 hours light/8 hours dark conditions. A small cotton ball soaked in selection solution was mounted on the infected area (“top selection”). Top selection solution contained 100 μM glyphosate, 1-2 mg/L 6-Benzylaminopurine and 1 g/L 2-(N-morpholino) ethanesulfonic acid (MES). The trays were covered with a dome to maintain high humidity. The cotton balls were changed 2 times weekly. Top selection lasted for 2 weeks. After one-week top selection, 300-500 μM glyphosate solution was watered into soil pots (“bottom selection”). The selection watering was done once a week, for at least 3 weeks.

### 2). ALS Selection to Generate Transgenic Events:

Selection for event regeneration: the plants were placed in a growth chamber under 16 hours light/8 hours dark conditions. A small cotton ball soaked in selection solution was mounted on the infected area (“top selection”). Top selection solution contained 3 μM bensulfuron-methyl, 1-2 mg/L 6-benzylaminopurine and 1 g/L 2-(N-morpholino) ethanesulfonic acid. The trays were covered with a dome to maintain high humidity. The cotton balls were changed 2 times weekly. Top selection lasted for 2 weeks. After one-week top selection, 1.5 μM Imazapyr solution was watered into soil pots (“bottom selection”). The selection watering was done once a week, for at least 3 weeks.

In both selection protocols, the putative events were first identified based on their growth and leaf morphology. The putative transgenic shoots grew fast and had normal leaves. The non-transgenic shoots were stunted, grew slowly or had small and narrow leaves. Leaves from fully transgenic shoots stay deep green and continue to grow without abnormal phenotype, while no transgenic leaves from chimeras appear in yellow or yellow-green sector.

### 6. Optimization for Selection and Recovery of Transgenic Plants Using Spay Application of Herbicides in Greenhouse

### 1) Glyphosate Selection to Generate Transgenic Events

After 2 day in Soy2 medium and 5 days in Soy2 medium with 100 μM glyphosate, the explants were transplanted to soil for further selection and development of transgenic shoots. The selection is performed for three weeks in GH by application with various concentrations of glyphosate using mounted cotton ball or daily liquid spay methods. The various concentrations of glyphosate are listed in Table 3. The recovered shoots from each treatment were subjected for Taqman assay to confirm the transgenic frequency. The application solution contains glyphosate, 1 g/L MES and BAP (2 mg./L for first week, and 1 mg/L for second and third weeks, or 2 mg/L or 1 mg/l for three weeks).

### 2) ALS Selection to Generate Transgenic Events

The explants were transplanted to soil for further selection and development of transgenic shoots after immersed in Soy2 medium for 2 day and in Soy2 medium with 2 μM bensulfuron-methyl for 5 days. The selection is performed for three weeks in GH by application with various concentrations of bensulfuron-methyl using mounted cotton ball or daily liquid spay methods. The various concentrations of bensulfuron-methyl are listed in Table 4. The recovered shoots from each treatment were subjected for Taqman assay to confirm the transgenic frequency. The application solution contains bensulfuron-methyl, 1 g/L MES and BAP (2 mg./L for first week, and 1 mg/L for second and third weeks, or 2 mg/L or 1 mg/l for three weeks). Alternatively, the transplanted explants in soil can be directly spayed with selection solution containing commercial herbicide Ecomazayr (1-8 μM isopropylamine salt of imazapyr), 1-2 mg/L BAP and 1 g/L MES for 3 weeks.

### 7. Dual Herbicide Selection:

The wounded explants were co-infected with Agrobacterium suspension containing a mixture of two Agrobacterium strains harboring 23093 or 18891 (OD A660 of approximately 0.6 for each strain) for 18 hours in dark at room temperature. After infection, the explants were removed from the Agrobacterium suspension and transferred to petri dishes for co-cultivation in an airtight plastic container. The co-cultivation plates were incubated for 4 days at 22±1° C. in the dark, with explants placed adaxial side up. After co-cultivation, the explants were immersed with liquid medium Soy 2 for two days followed by liquid medium Soy2 with both 100 μM Glyphosate and 2 μM bensulfuron-methyl for 5 days. The explants were then transferred to soil and were mounted in the wounded region with a small cotton ball containing selection solution contained 100 μM glyphosate and 3 μM bensulfuron-methyl. The explants in the soil in trays were covered with a dome to maintain high humidity. The cotton balls were changed 2 times per week for two weeks, then spay daily with selection solution containing 100 μM glyphosate and 3 μM bensulfuron-methyl for additional one week. Alternatively, the explants can also be sprayed with selection solution contained 100 μM glyphosate and 3 μM bensulfuron-methyl. Regenerated shoots then were sampled for Taqman assay.

8. Application to Diverse Soybean Germplasms from Different Maturity Group

### 1) Glyphosate Application for EPSPS Selection

In addition to elite line 06KG218440 (RM 5.5), a group of 8 diverse soybean elite lines from various maturity groups of RM1.9 to RM8.2 listed in Table 5 are selected for transformation. The group also represents a huge variation of transformability in tissue culture-based transformation method, transformation frequency ranges from 2% to 32%. After co-cultivation, the explants were immersed in Soy2 without glyphosate for 2 days followed by pre-selection in Soy2 medium with 100 μM glyphosate for 5 days. Then, the infected area of explant was mounted with a small cotton ball soaked with selection solution contained 100 μM glyphosate after the explants were transferred to soil in 2-inch pots. The explants in the soil in trays were covered with a dome to maintain high humidity. The cotton balls were changed 2 times per week for two weeks, then 300 μM glyphosate solution was watered into soil pots for additional one week. Regenerated shoots then were sampled for Taqman assay.

### 2) Bensulfuron-Methyl Application for ALS Selection

In addition to elite line 06KG218440 (RM 5.5), a group of 13 diverse soybean elite lines from various maturity groups of RM1.9 to RM8.6 listed in Table 5 are selected for transformation. The group also represents a huge variation of transformability in tissue culture-based transformation method, transformation frequency ranges from 1% to 33%. After co-cultivation, the explants were immersed in Soy2 without bensulfuron-methyl for 2 days followed by pre-selection in Soy2 medium with 2 μM bensulfuron-methyl for 5 days. Then, the explants were transferred to soil in 2-inch pots in trays. The explant was sprayed with selection solution contained 3 μM bensulfuron-methyl, 1-g/L MES and 2 mg/L BAP for first week, followed by selection solution contained 3 μM bensulfuron-methyl, 1 g/L MES and 1 mg/L BAP two weeks. The explants in the soil in trays were covered with a dome to maintain high humidity. Regenerated shoots then were sampled for Taqman assay.

### 9. Transgene Inheritance

The immature seeds are examined for the expression of AmCyan gene from a total of 75 non-backbone, single copy transgenic events, representing 5 independent transformation experiments using ALS or EPSPS selectable markers. The immature seeds from randomly picked-10 pots per plants are harvested from transgenic events in greenhouse and seed coats are carefully removed from cotyledons and then the immature embryos are observed under microscope for fluorescence expression of AmCyan gene.

Results of Soybean Transformation Experiments from the Above Described Method
1. The results of primary Taqman analysis on recovered plants four weeks after infection with Agrobacterium harboring binary vector 23093 are summarized in Table 1. The results show a comparison of treatment conditions that results in significant improvement of transformation frequency. Using liquid medium for pre-selection, transformation frequency was significantly improved up to 45%. The condition of 2-day pre-treatment with the medium without glyphosate followed by 5-day preselection with 100 μM glyphosate improves the transformation frequency. Overall, each of the tested methods with selection could produce transformation events.

TABLE 1
Transformation frequency comparison under different
selection conditions using EPSPS as selectable marker

| Treatment ID | A | B | C | D | E |
|---|---|---|---|---|---|
| Treatment | Liquid pre- | Liquid pre- | Paper towel | Growth in | Liquid pre- |
| condition | selection, 2-day | selection, 7 | with 7-day in | paper towel | selection, 2-day |
| | in soy2 medium | days in soy2 | soy2 medium | and transplant | in soy2 medium |
| | without glyphosate, | medium with | without glyphosate, | to GH without | without glyphosate, |
| | 5 days in soy2 | 75 μM | 7 days in soy2 | selection | 5 days in soy2 |
| | medium with | glyphosate | medium with | | medium with |
| | 75 μM | | 75 μM | | 100 μM |
| | glyphosate | | glyphosate | | glyphosate |
| Elite variety | 06KG218440 | | | | |
| Construct(s) | 23093 | | | | |
| Selectable | EPSPS | | | | |
| marker | | | | | |
| Number of | 66 | 60 | 30 | 60 | 251 |
| Explants | | | | | |
| Total Number | 20 | 15 | 1 | 0 | 114 |
| of Events | | | | | |
| Transformation | 30.3 | 25.0 | 3.3 | 0.0 | 45.4 |
| Frequency (%) | | | | | |
| Number of low | 6 | 5 | 1 | 0 | 43 |
| Copy Events | | | | | |
| Number of low | 5 | 4 | 1 | 0 | 33 |
| Copy no | | | | | |
| Backbone | | | | | |
| Events | | | | | |
| Number of | 5 | 7 | 0 | 0 | 32 |
| Backbone + | | | | | |
| Events | | | | | |

2. The results of primary Taqman analysis on recovered plants four weeks after infection with Agrobacterium harboring binary vector 18891 are summarized in Table 2. The results show transformation frequency was as high as more than 51% using ALS as selectable marker. The transformation frequency was significantly improved by combination 2-day recovery and 5-day pre-selection with 1-2 μM Bensulfuron-methyl.

TABLE 2
Transformation frequency comparison under different
selection conditions using ALS as selectable marker

| Treatment ID | A | B | C |
|---|---|---|---|
| Treatment condition | Liquid pre-selection, 2- | Liquid pre-selection, 7 | Liquid pre-selection, 2- |
| | day in soy2 medium | days in soy2 medium | day in soy2 medium |
| | without Bensulfuron- | with 2 μM | without Bensulfuron- |
| | methyl, 5 days in soy | Bensulfuron-methyl | methyl, 5 days in soy |
| | medium with 1 μM | | medium with 5 μM |
| | Bensulfuron-methyl | | Bensulfuron-methyl |
| Variety | 06KG218440 | | |
| Construct(s) | 18891 | | |
| Selectable marker | ALS | | |
| Number of Explants | 130 | 124 | 130 |
| Total Number of Events | 67 | 58 | 36 |
| Transformation | 51.5 | 46.8 | 27.7 |
| Frequency (%) | | | |
| Number of low Copy | 26 | 27 | 17 |
| Events | | | |
| Number of low Copy no | 23 | 26 | 13 |
| Backbone Events | | | |
| Number of Backbone + | 18 | 8 | 9 |
| Events | | | |

3. Optimization of glyphosate concentrations and application methods for EPSPS selection

To obtain an optimal selection conditions and methods for EPSPS selection, a side-by-side comparison experiment was conducted. The results are summarized in Table 3. Transgenic events can be recovered without selection in Greenhouse at 18% transformation frequency with more than 80% escape rate. By using cotton ball method for application of 175 μM glyphosate, high transformation frequency (˜48%) with high single copy event rate (>50%) was obtained without any escape event. Similar results can also be achieved by daily spay of 150 μM or more of glyphosate with few escapes.

TABLE 3
Effects of application methods and glyphosate concentrations on
transformation frequency, escape rate and single copy event rate

| | | | | | | | Number | |
|---|---|---|---|---|---|---|---|---|
| | | | Number | | | | events | |
| | Glyphosate | Number | of | Number | | | of | Single |
| Application | concentration | of | plants | of | Transformation | Escape | single | Copy |
| Method | (μM) | explants | sampled | events | Frequency | rate | copy | rate |
| cotton ball | 100 | 48 | 17 | 16 | 33.3% | 2.1% | 8 | 50.0% |
| cotton ball | 125 | 48 | 25 | 25 | 52.1% | 0.0% | 10 | 40.0% |
| cotton ball | 150 | 48 | 24 | 24 | 50.0% | 0.0% | 8 | 33.3% |
| cotton ball | 175 | 48 | 23 | 23 | 47.9% | 0.0% | 12 | 52.2% |
| Spray | 100 | 48 | 12 | 11 | 22.9% | 2.1% | 6 | 54.5% |
| Spray | 125 | 48 | 16 | 13 | 27.1% | 6.3% | 6 | 46.2% |
| Spray | 150 | 48 | 21 | 20 | 41.7% | 2.1% | 11 | 55.0% |
| Spray | 175 | 48 | 25 | 22 | 45.8% | 6.3% | 9 | 40.9% |
| Spray | 200 | 48 | 23 | 20 | 41.7% | 6.3% | 6 | 30.0% |
| Spray | 250 | 48 | 17 | 15 | 31.3% | 4.2% | 9 | 60.0% |
| Spray | 300 | 48 | 24 | 23 | 47.9% | 2.1% | 9 | 39.1% |
| Spray | 0 | 144 | 144 | 27 | 18.8% | 81.3% | 15 | 55.6% |

4. Optimization of bensulfuron-methyl concentrations and application methods for ALS selection

To obtain an optimal selection conditions and methods for ALS selection, a side-by-side comparison experiment was conducted. The results are summarized in Table 3. Transgenic events can be recovered without selection in Greenhouse at more than 18% transformation frequency with 73% escape rate. No escape event was recovered when applying 3 μM or higher concentrations of bensulfuron-methyl. By using cotton ball method for application of bensulfuron-methyl, high transformation frequency (˜40%) with high single copy event rate (50%) was obtained. Similar high transformation frequency can also be achieved by daily spay of 3 μM bensulfuron-methyl with more than 60% single copy rate.

TABLE 4
Effects of application methods and bensulfuron-methyl concentrations
on transformation frequency, escape rate and single copy event rate

| | Bensulfuron- | | | | | | Number | Number |
|---|---|---|---|---|---|---|---|---|
| | methyl | Number | Number | Number | | | of single | of single |
| Application | concentration | of | of plants | of | | Escape | copy | copy |
| Method | (μM) | explants | sampled | events | TF | rate | events | rate |
| Spray | 3 | 48 | 18 | 18 | 37.5% | 0.0% | 11 | 61.1% |
| Spray | 4 | 48 | 10 | 10 | 20.8% | 0.0% | 8 | 80.0% |
| Spray | 5 | 48 | 10 | 10 | 20.8% | 0.0% | 7 | 70.0% |
| Spray | 6 | 48 | 10 | 10 | 20.8% | 0.0% | 4 | 40.0% |
| Spray | 7 | 48 | 6 | 6 | 12.5% | 0.0% | 5 | 83.3% |
| Spray | 8 | 48 | 11 | 11 | 22.9% | 0.0% | 7 | 63.6% |
| Spray | 9 | 48 | 12 | 12 | 25.0% | 0.0% | 8 | 66.7% |
| Spray | 10 | 48 | 8 | 8 | 16.7% | 0.0% | 4 | 50.0% |
| cotton ball | 3 | 40 | 16 | 16 | 40.0% | 0.0% | 8 | 50.0% |
| cotton ball | 6 | 48 | 13 | 13 | 27.1% | 0.0% | 7 | 53.8% |
| cotton ball | 9 | 48 | 9 | 9 | 18.8% | 0.0% | 0 | 0.0% |
| control | 0 | 180 | 126 | 34 | 18.9% | 73.0% | 19 | 55.9% |

5. Application of glyphosate or bensulfuron-methyl to diverse soybean germplasms from different maturity groups

The results are summarized in Table 5 from analysis of Taqman assay on transgenic plants from diverse soybean germplasms five weeks after the infection of explants with Agrobacterium harboring 23093 or 18891. The results show the method applicable to diverse soybean germplasms from different maturity groups with high transformation frequency, a genotype independent transformation. Specifically, transformation frequency is significantly improved in 5 to 10-fold for those recalcitrant lines in tissue culture-based transformation method.

TABLE 5
The results of glyphosate or bensulfuron-methyl to diverse soybean germplasms

| | | EPSPS selectable | | ALS selectable marker | |
|---|---|---|---|---|---|
| | Relative | marker (23093) | | (18891) | |
| | Maturity | | Sigle | | Single |
| Elite | group | Transformation | copy rate | Transformation | copy rate |
| variety ID | (RM) | frequency (%) | (%) | frequency (%) | (%) |
| OW0902057-3 | 1.9 | 37.9 | 33.3 | 35.4 | 35.2 |
| NE1306802 | 2.1 | NA | NA | 22.9 | 36.4 |
| NE1306405 | 2.5 | NA | NA | 22.9 | 45.5 |
| NE0800097 | 2.7 | 17.7 | 50.0 | 30.0 | 60.0 |
| CA1504095 | 5.8 | 18.2 | 58.3 | 33.9 | 45.0 |
| BS1602695 | 6.0 | 12.7 | 11.1 | 39.3 | 58.3 |
| CA1503606 | 6.0 | NA | NA | 27.1 | 76.9 |
| BS1601456 | 6.5 | 28.6 | 28.6 | 41.7 | 45.0 |
| BC1601020 | 6.6 | NA | NA | 22.9 | 72.7 |
| UB1500442 | 7.0 | 11.7 | 42.9 | NA | NA |
| LR1500228 | 7.6 | NA | NA | 24.6 | 56.3 |
| BW1600439 | 8.0 | 36.2 | 72.0 | 21.8 | 58.3 |
| BW1601085 | 8.2 | 36.2 | 40.0 | 31.3 | 60.0 |
| BW1601120 | 8.6 | NA | NA | 29.2 | 50.0 |

NA: not applicable.

6. Dual herbicide selection

Table 6 summarized the results from co-transformation of two selectable markers, EPSPA and ALS, harbored in different Agrobacterium strains after application of dual herbicide selection. The co-transformation frequency is more than 30% while overall transformation frequency is more than 50% in dual herbicide selection.

TABLE 6
Co-transformation frequency of 23093 and 18891 using dual
herbicide selection in soybean elite line 06KG218440

| | | | | | | Transformation Frequency (%) | | | |
|---|---|---|---|---|---|---|---|---|---|
| | | | | | | | Co- | | |
| | Number | Number of events | | | | | transformation | | |
| Sel. | of | | ALS + | ALS | EPSPS | | (ALS + | ALS | EPSPS |
| Marker(s) | explants | Total | EPSPS | only | only | Overall | EPSPS) | only | only |
| ALS/EPSPS | 284 | 145 | 86 | 49 | 10 | 51.1 | 30.3 | 17.3 | 3.5 |

7. Inheritance analysis of transgene

The transgene AmCyan is present in both 23093 and 18891. All transgenic plants generated from both constructs will carry visible marker gene, AmCyan, and selectable marker gene, EPSPS or ALS. The inheritance of transgene can be demonstrated by observation the expression of AmCyan gene, the CFP fluorescence, under UV light in immature embryos of transgenic plants. Table 7 summarized the results of CFP expression examined in immature embryos from progeny of a total of 75 transgenic events containing single copy gene without vector backbone. 41 out of 45 transgenic events were CFP positive in immature embryos using glyphosate selection, while 29 out of 30 transgenic events were CFP positive in progeny using bensulfuron-methyl selection. The results demonstrate the inheritable transmission of the transgenes from T0 to T1 generation at high efficiency.

TABLE 7
Transgene inheritance in T1 progeny

| Treatment | | | | | Number | Average | |
|---|---|---|---|---|---|---|---|
| ID (from | | | | Number | of pots/ | number | CFP |
| Tables 1 | | | Selectable | of Events | events | of seeds/ | positive |
| and 2) | Variety | Construct(s) | marker | analyzed | analyzed | events | events |
| A | 06KG218440 | 23093 | EPSPS | 12 | 10 | 21 | 11 |
| B | | | | 12 | 10 | 20 | 10 |
| E | | | | 21 | 10 | 22 | 20 |
| A | | 18891 | ALS | 15 | 10 | 18 | 15 |
| B | | | | 15 | 10 | 24 | 14 |
| Total | | | | 75 | 50 | 21 | 70 |

8. Application of commercial herbicide Ecomazayr 2SL

An experiment was conducted to compare the selection effectiveness in greenhouse between BSU and commercial herbicide Ecomazayr for ALS selectable marker using current transformation method. The results summarized in Table 8 shows that the commercial herbicide Ecomazayr is as effective as BSU and can be used in greenhouse selection for recovery of ALS transgenic plants in current transformation method

TABLE 8
Comparison of selection effectiveness between
BSU and commercial herbicide Ecomazayr 2SL

| | | | | | Number | | |
|---|---|---|---|---|---|---|---|
| | | Number | Number | Transformation | of single | Single | Number |
| Greenhouse | | of | of | frequency | copy | copy rate | of |
| selection | | explants | events | (%) | events | (%) | escapes |
| BSU | 3 | 56 | 12 | 21.4% | 5 | 41.7% | 0 |
| (μM) | | | | | | | |
| imazapyr | 1 | 56 | 15 | 26.8% | 6 | 40.0% | 1 |
| (μM) | 2 | 56 | 12 | 21.4% | 7 | 58.3% | 0 |
| | 3 | 56 | 20 | 35.7% | 8 | 40.0% | 0 |
| | 4 | 56 | 14 | 25.0% | 6 | 42.9% | 0 |
| | 5 | 56 | 8 | 14.3% | 2 | 25.0% | 0 |
| | 6 | 56 | 10 | 17.9% | 6 | 60.0% | 0 |
| | 7 | 52 | 10 | 19.2% | 6 | 60.0% | 0 |
| | 8 | 54 | 10 | 18.5% | 6 | 60.0% | 0 |

9. Optimization of in-vitro selection and greenhouse selection process

An experiment was executed to improve and simplify the selection process and condition. Soy 3 medium was developed to improve transformation efficiency at in-vitro recovery and pre-selection stage. Soy3 medium contains all Soy2 medium components but replaced 2 mg/L BA with 1 mg/L BA and 1 mg/L zeatin riboside. In greenhouse selection, one step formulated selection solution for 3 weeks was replaced 2 steps selections for 3 weeks. The results demonstrated that transformation frequency was increased from 21% with two steps greenhouse selection to 28.6% in one step selection with 1 or 2 mg/L BA. The transformation frequency was improved 50% when Soy3 medium was used in in-vitro recovery and preselection with one step greenhouse selection (treatment C vs. D) while single copy rate remains similar.

TABLE 9
Optimization of in-vitro and greenhouse selection process

| Treatment | A | B | C | D | E | F |
|---|---|---|---|---|---|---|
| In-vitro recovery | Soy2 | Soy2 | Soy2 | Soy3 | Soy2 | Soy3 |
| In vitro | Soy2 + | Soy2 + | Soy2 + | Soy3 + | Soy2 + | Soy3 + |
| preselection | 2 μM BSU | 2 μM BSU | 2 μM BSU | 2 μM BSU | 2 μM BSU | 2 μM BSU |
| Greenhouse | 3 μM BSU + | 2 μM BSU + | 2 μM BSU + | 2 μM BSU + | 2 μM BSU + | 2 μM BSU + |
| selection | 2 mg/L | 0 mg/L | 1 mg/L | 1 mg/L | 2 mg/L | 2 mg/L |
| | BA | BA | BA | BA | BA | BA |
| | 3 μM BSU + | | | | | |
| | 1 mg/L | | | | | |
| | BA | | | | | |
| Number of | 56 | 168 | 168 | 56 | 168 | 56 |
| explants | | | | | | |
| Number of | 12 | 34 | 48 | 24 | 48 | 19 |
| events | | | | | | |
| Transformation | 21.4 | 20.2 | 28.6 | 42.9 | 28.6 | 33.9 |
| frequency (%) | | | | | | |
| Number of | 5 | 21 | 26 | 16 | 31 | 5 |
| single copy | | | | | | |
| events | | | | | | |
| Single copy | 41.7 | 61.8 | 54.2 | 66.7 | 64.6 | 26.3 |
| rate (%) | | | | | | |
| Number of | 0 | 1 | 0 | 1 | 2 | 0 |
| escapes | | | | | | |

### Example 3 Biolistic Transformation of Soybean with the Semi-In-Planta Method

1) Transformation with DNA

About 1×10¹⁰˜10¹¹ molecules of DNA of interest (vector 23092 or 18891 with 22296) are added to a tube of 50 μl of prepared gold-glycerol slurry and mix well by vortexing, followed by adding equal volume of cold 2.5 M CaCl₂ to a final concentration of 1.25 M CaCl₂ and 10 μl of cold 0.1M spermidine then mixing immediately. After keeping the DNA-gold particle mixture in ice for at least 30 min to precipitate DNA onto gold particles, spin the DNA-gold particle mixture at 14,000 rpm for 5 seconds and remove the supernatant carefully with pipette. After adding 200 μl of cold 100% (200 proof) ethanol and mix well, spin the DNA-gold particle mixture at 14,000 rpm for 5 seconds and remove the supernatant carefully with pipette. Then re-suspend the mixture in about 50 μl of cold 100% (200 proof) ethanol for 9 shots. Pipette 5 μl of the mixture evenly onto the center of prepared macrocarrier-holder unit and allow to dry before bombardment.

Explants were prepared by carefully removing and discarding the seed coat, one of the cotyledons and both primary leaves from the soaked seed. About 30 explants were arranged in one circle with exposed shoot apical region facing to bombardment direction in a target plate containing MS basal medium 2 mg/L BAP. Each target plate is bombarded three times at 1100 or 1300 psi under the vacuum at 27.5 mm Hg using the PDS Helium-1000 device. After bombardment, the plates containing bombarded explants were cultured for 2-3 days at 24° C. under 16 hours light/8 hours regimen, and >80 μE/m2/s. The explants were then transferred in adequate Soy2 medium with 100 μM glyphosate or 2 μM BSU for 5-7 days. After the explants were transplanted to soil, the selection was performed in a growth chamber or greenhouse by application with selection solution containing 100 μM glyphosate or 2 μM BSU, 1 mg/L MES and 2 mg/LBAP for three weeks. The recovered shoots from each treatment were subjected for Taqman assay to confirm the transgenic frequency.

The results summarized in Table 9 demonstrated that the described transformation method can be applied in Biolistic-mediated transformation for transgenic plant recovery. The transgenic plants confirmed by Taqman assay were generated from bombarded explants using BSU and glyphosate selection by different gene delivery conditions and selection schemes in less than 6 weeks after bombardment. It is applicable to both transformation and co-transformation of gene of interested.

TABLE 10
Recovery of transgenic events from biolistic bombardment
of plasmid DNA using ALS or EPSPS selectable marker

| Construct(s) | 18891, 22296 | 18891, 22296 | 22296 | 23093 |
|---|---|---|---|---|
| Selectable | ALS | ALS | ALS | EPSPS |
| marker | | | | |
| psi | 1100 | 1100 | 1350 | 1100 |
| In vitro pre- | 2 μM BSU | 2 μM BSU | 2 μM BSU | 100 μM |
| selection | | | | glyphosate |
| In greenhouse | 3 μM BSU | 3 μM BSU | 1 μM BSU | 300 μM |
| selection | | | | glyphosate |
| number of | 68 | 108 | 307 | 168 |
| explants | | | | |
| Total number | 3 | 4 | 18 | 1 |
| of Events | | | | |
| Escapes | 0 | 0 | 3 | 0 |
| Transformation | 4.41 | 3.7 | 5.86 | 0.6 |
| Frequency (%) | | | | |

2) Transformation with Ribonucleoprotein (RNP)

To prepare RNP complex, both AsCas12a nuclease and gRNA (rLbgRNACas12aGmFAD2-03, TAATTTCTAC TAAGTGTAGA TGAACCCTTG AGAGAGGCTT CTTC) were prepared with nuclease-free water to desired volume before used. AsCas12a and crRNA can be purchased from IDT. 0.3 nmol of AsCas12 nuclease (5 μl of 60 μM) and 0.3 nmol of crRNA (6 μl of 50 μM) were mixed gently to a total volume of 11 μl and incubated at room temperature for 10 min. A tube of 50 μl of prepared gold slurry was prepared with 1 mg 0.6 μm gold particles in 50 μl nuclease-free water after gold particles were sonicated and sterilized with 100% ethanol. The RNP complexes were then added to the 50 μl gold slurry and then mixed gently. Optionally, the plasmid DNA of interest containing selectable marker (1×10¹⁰ molecules DNA of 22296) were also added with the RNP complexes to allow recovery of edited events. After incubated on ice for 10 min, The RNP/DNA coated gold particles were then centrifuged at 8,000×g (˜11,000 rpm with Eppendorf microfuge 5410) for 40 s and supernatant was removed. The pellet was resuspended with 30 μl of sterile water by brief sonication, and then loaded onto a macrocarrier (10 μl each) followed by air dry in the laminar flow hood for 2 hours.

Explants were prepared by carefully removing and discarding the seed coat, one of the cotyledons and both primary leaves from the soaked seed. About 30 explants were arranged in one circle with exposed shoot apical region facing to bombardment direction in a target plate containing MS basal medium 2 mg/L BAP. Each target plate is bombarded three times at 1100 or 1300 psi under the vacuum at 27.5 mm Hg using the PDS Helium-1000 device. After bombardment, the plates containing bombarded explants were cultured for 2-3 days at 24° C. under 16 hours light/8 hours regimen, and >80 μE/m2/s. The explants were then transferred in adequate Soy2 medium with 100 μM glyphosate or 2 μM BSU for 5-7 days. After the explants were transplanted to soil, the selection was performed in GH by application with selection solution containing 100 μM glyphosate or 2 μM BSU, 1 mg/L MES and 2 mg/LBAP for three weeks. The recovered shoots from each treatment were subjected for Taqman assay to confirm the transgenic and editing frequency.

An experiment on editing soybean FAD2 gene was conducted by co-transformation of ALS selectable marker DNA (22296), gRNA (rLbgRNACas12aGmFAD2-03, TAATTTCTAC TAAGTGTAGA TGAACCCTTG AGAGAGGCTT CTTC) and LbCas12a ribonucleoprotein. After plants were recovered in 6 weeks, leaf tissue from the events were sampled and subjected for Taqman assay for analysis. The results demonstrated 7 putative editing events were identified from 780 bombarded explants.

### Example 4 Transformation of Wild Glycine Species Glycine tomentella with the Semi-In-Planta Method

The wild Glycine species Glycine tomentella is a rich source of disease resistance (R) genes, for example soybean cyst nematode and Asian soybean rust. Transformation method for G. tomentella is very useful for R gene identification and validation. The seeds of Glycine tomentella were treated with sulfuric acid for 40 minutes before sterilization. After sterilized with 15% Clorox for 15 minutes, the seeds were washed with sterilized water three time and then germinated in dark or under light on MS basal medium containing 2 mg/L BA and 30 g/L sucrose. The germinated seedling was carefully removed and discarded one cotyledon and two primary leaves. The region containing the epicotyl, apical meristem and the cotyledonary node were wounded by making several cuts with the sharp end of scalpel blade as describes above for soybean transformation. Following the same protocol described above for soybean Agrobacterium transformation in vitro and selection conditions in GH, the leaves from recovered plants were sampled and subjected for Taqman assay to confirmation if transgenic events.

An experiment was performed to transform G. tomentella with Agrobacterium harboring vector 18891. Following the above described process, Plants were recovered 4 weeks after infection with Agrobacterium. Leave tissues from the recovered plants were sampled and subjected for Taqman assay. The results demonstrated that one transgenic event was identified from 25 explants infected with Agrobacterium.

### Example 5 Transformation of Sunflowers (Helianthus annuus) with the Semi-In-Planta Method

The pericarp of sunflower seeds was carefully removed and discarded. The seeds were then soaked in water for 2-4 hours before sterilization. After sterilized with 20% Clorox for 15 minutes, the seeds were washed with sterilized water three time and then germinated in dark or under light on MS basal medium containing 2 mg/L BA and 30 g/L sucrose. The germinated seedling was carefully removed and discarded one cotyledon and two primary leaves. The region containing the epicotyl, apical meristem and the cotyledonary node were wounded by making several cuts with the sharp end of scalpel blade as describes above for soybean transformation. Following the same protocol described above for soybean Agrobacterium transformation in vitro and selection conditions in GH, the leaves from recovered plants were sampled and subjected for Taqman assay to confirmation if transgenic events.

### Example 6. Enhancement of Transformation Efficiency of Semi-In-Planta Method in Recalcitrant Crop by Co-Transforming with a Vector Expressing Morphogenic Factor(s) or Developmental Regulator(s)

### 1) Target Tissue Preparation

Sterilized or unsterilized mature or immature seeds, preferably sterilized mature seeds, of recalcitrant varieties of soybean and corn, and recalcitrant crops such as sunflower, cotton, watermelon or sugar beet are used. Prior to Agrobacterium inoculation, soak seeds in water or other liquid medium for 4-48 hours, preferably overnight. Alternatively, sterilized seeds can also be germinated in solid medium overnight. The seeds are incubated at 22-24° C. for at least 16-20 hours.

The seed coat is removed from the soaked or germinated seed. The seed is then wounded by carefully cutting into region comprising the epicotyl and shoot apical meristem (e.g., with a scalpel blade) without completely detaching the hypocotyl from cotyledons. A preferred alternative method is to carefully remove and discard one of the cotyledons, and then wound the region comprising the epicotyl, apical meristem and cotyledonary node (e.g., with the sharp end of a scalpel blade). As another alternative, the primary leaves and one of the cotyledons can be removed before wounding the region. Optionally, prepared explants can be further wounded by other methods such as sonication or whisker-mediated abrasion.

### 3) Agrobacterium Suspension Preparation

Different Agrobacterium tumefaciens strain can be used for transformation, preferably such as EHA101 or Chry5 (various versions of each strain may be used, including recA−). Agrobacterium harbors binary vectors containing selectable marker and gene(s) of interest. Example constructs contain EPSPS or Acetolactate synthase (ALS) selectable markers. Agrobacterium culture is streaked from −80° C. from glycerol stock onto plates containing appropriate antibiotics and grown in a 22-28° C. incubator, preferably, 23° C. Before inoculation with explants, Agrobacterium cells are collected from the plate, uniformly suspended in liquid infection medium in a sterile disposable 50 ml centrifuge tube, and diluted to OD A660 of approximately 0.20 to 1.0, preferably an OD of approximately 0.3 to 0.6. Acetosyringone is added to induce virulence gene expression. Preferably, dithiothreitol (DTT) is added.

A second Agrobacterium strain is also included. This second Agrobacterium is transformed with a binary vector containing an expression cassette driving the expression of a morphogenic factor (MF) or a developmental regulator (DR) such as Baby Boom (BBM), Wuschel (WUS/Wox), Growth-Regulating Factor (GRF), Growth-Regulating Factor 4 (GRF4) and its cofactor GRF-Interacting Factor 1 (GIF1), Shoot Meristemless (STM) or Isopentenyl Transferase (IPT). Expression of the MF/DR improves transformation of recalcitrant plants through de novo meristem induction. A second expression cassette drives 1) pollen specific expression of barnase selecting against gametes with the co-transformed MF/DR transgene thereof, or 2) fluorescent marker genes expressed in seeds, embryos or seedlings allowing the identification and removal of events with the MF/DR transgene in the gene of interest (GOI)/genome edited (GE) progeny.

### 4) Infection and Incubation

The wounded explants are immediately infected with Agrobacterium by immersing them with the Agrobacterium suspension, and then incubated for at least 30 minutes or up to overnight in dark at room temperature. Alternatively, the infection of explants can also be carried out in the presence of Agrobacterium suspension, e.g., by adding the Agrobacterium suspension on the wounding region first, and then wounding the explants; or by dipping a scalpel blade in Agrobacterium suspension and then using the blade to wound the explants, or by directly wounding the explants in Agrobacterium suspension. The explants/Agrobacterium mixture can also be treated with heat shock, sonication or vacuum to enhance infection. After infection, the explants are removed from the Agrobacterium suspension and transferred to incubation on a petri dish or solid medium within a plastic container. The incubation plates are incubated for 3 to 6 days (preferably 4-5 days) at 21-23° C. in the dark, with explants placed adaxial side up.

### 5) Recovery and Optional Pre-Selection In Vitro

After incubation, liquid medium, with or without corresponding selection agent, is added to just immerse the explants in incubation plate or vessel. The cultures are then incubated at 23-28° C., preferable 25° C. under light for up to 4 weeks before being transplanted to soil for further selection and transgenic shoot regeneration and recovery. Selection agent for EPSPS gene is 25-500 μM glyphosate, preferably 100 μM. Selection agent for ALS gene is 0.01-1 μM Bensulfuron-methyl, preferably at 0.1-0.3 μM. Other corresponding selection agents can also be used.

### 6) Selection and Recovery of Transgenic Plants

After recovery and optional pre-selection, the infected explants are transplanted to soil for further selection and development of transgenic shoots. The plants are placed in a growth chamber under 16 hour light/8 hour dark conditions in trays. A small cotton ball soaked in selection solution is mounted on the infected area (“top selection”) of each plant. The trays are covered with a dome to maintain high humidity. The cotton balls are changed 1-2 times weekly. Top selection can be performed for up to 2 weeks. After one week of top selection, selection solution can be watered into soil pots (“bottom selection”). Alternatively, just bottom or just top selection with herbicide may be used. For top selection, the plants are sprayed with sublethal level of herbicide to suppress growth of the non-transformed tissues and allow growth of transformed shoots. For bottom selection, selection is done through drenching (supplying the sublethal level of herbicide selection agent in water) once a week, for 3-5 weeks.

### 7) Transgenic Plant Identification and Confirmation

Transgenic shoots develop without apparent abnormal phenotype and can be distinguished from chimeras which are expected to have yellow and green leaves in developed shoots or a chimeric sector in a transgenic leaf. The true transgenic shoots can be confirmed by molecular analysis such as Taqman analysis. Two to three leaves are sampled from different sets of trifoliate in one transgenic shoot and subjected to analysis the presence of selectable marker, binary vector backbone and gene-of-interest.

### 8) Inheritance of Transgene

Seeds harvested from independent transgenic shoots are germinated and leaves are sampled for molecular analysis such as Taqman analysis for the presence of selectable marker, binary vector backbone and gene-of-interest.

### REFERENCES

U.S. Pat. No. 5,376,543
U.S. Pat. No. 5,015,580
U.S. Pat. No. 7,002,058
US20020073445
US2003046733
US2004034889
US2005268357
US20040237133
WO0042207
WO05121345
CN103667342A
CN106399359A
Li et al. Optimization of Agrobacterium-Mediated Genetic Transformation System of Soybean Cotyledonary Node with Non Tissue-Culture. 2013. Journal of Plant Genetic Resources, Vol. 13, No. 5, pp. 789-797.
Janani et al. Construction and transformation of peroxisome proliferator activated receptor gamma (RnPPARγ) gene using Agrobacterium tumefaciens into Glycine max L. Merr. 2019. Gene Reports, Vol. 16, p. 100427.
Mangena et al. Challenges of In Vitro and In Vivo Agrobacterium-Mediated Genetic Transformation in Soybean, Soybean—The Basis of Yield, Biomass and Productivity, Minobu Kasai, IntechOpen, DOI: 10.5772/66708. 2017. Available from: www.intechopen.com/books/soybean-the-basis-of-yield-biomass-and-productivity/challenges-of-in-vitro-and-in-vivo-agrobacterium-mediated-genetic-transformation-in-soybean
Soto et al. Efficient particle bombardment-mediated transformation of Cuban soybean (INCASoy-36) using glyphosate as a selective agent. 2017. Plant Cell, Tissue and Organ Culture, Vol. 128, No. 1, pp. 187-196.

1. A method of producing a chimeric plant with at least one transgenic shoot, comprising:
a) Providing a plant comprising an axillary meristem and a shoot apical meristem,
b) Removing or wounding at least part of the axillary meristem to produce a wounded axillary meristem region,
c) Contacting the wounded axillary meristem region with a heterologous polynucleotide and/or heterologous protein under conditions where the heterologous polynucleotide and/or heterologous protein enters wounded axillary meristem region,
d) Removing the shoot apical meristem or suppressing the growth of the shoot apical meristem at the same time as step b) or step c) or after step c) to produce a wounded explant,
e) Culturing the wounded explant in a medium in vitro to promote cell proliferation and regeneration, and
f) Growing the wounded explant in a proper medium and applying selection agent to the resulting plant in planta to select for a transgenic shoot.

2. The method of claim 1, wherein the wounding is performed by a method comprising cutting, piercing, crushing, pressure, sonication, or centrifugation.

3. The method of claim 1, wherein the seed is a dicot seed and step b) comprises (i) wounding at least part of the epicotyl and at least part of the cotyledonary node or (ii) wounding at least part of the epicotyl, at least part of the shoot apical meristem, and least part of the cotyledonary node.

4. The method of claim 1, wherein the seed is a monocot seed and step b) comprises wounding at least part of the coleoptile, at least part of the shoot apical meristem, at least part of the leaf primordia, and at least part of the leaf axillary region.

5. The method any-ene of claim 1, wherein the method further comprises removing a cotyledon from the explant.

6. The method of claim 5, wherein the seed is a dicot seed and the method further comprises removing one or both cotyledons from the explant, optionally wherein the dicot seed is a soy seed, a tobacco seed, a bean seed, a sunflower seed, a tomato seed or a pepper seed.

7. The method of claim 1, wherein the method further comprises removing at least one primary leaf from the explant.

8. The method of claim 1, wherein the method further comprises generating a plant from the wounded explant.

9. The method of claim 1, step c) comprises contacting the wounded axillary meristem region with a heterologous polynucleotide, wherein the heterologous polynucleotide comprises a selectable marker and wherein the method further comprises contacting the wounded explant or a plant or plant part generated from the wounded explant, or a combination thereof, with a selection agent to eliminate or reduce untransformed tissue.

10. The method of claim 9, wherein the contacting with the selection agent comprises adding the selection agent to a medium in which the wounded explant is maintained.

11. The method of claim 1, wherein the applying selection agent to the resulting plant in planta comprises (i) adding the selection agent to a medium in which the plant is maintained, (ii) spraying the plant with the selection agent, or (iii) applying the selection agent to the wounded area of the explant or the corresponding area of the plant, or a combination thereof.

12. The method of claim 11, wherein applying selection agent to the plant in planta comprises (i) adding the selection agent to a medium in which the plant is maintained, (ii) spraying the plant with the selection agent, or (iii) applying the selection agent to the wounded area of the explant or the corresponding area of the plant, optionally wherein (i) occurs for up to 4 weeks, (ii) occurs for up to 2 weeks and (iii) occur for up to 5 weeks.

13. The method of claim 12, wherein step (i) is performed prior to step (iii).

14. The method of claim 12, wherein at least part of step (i) is performed at the same time as at least part of step (iii).

15. The method of claim 9, wherein the selection agent is an herbicide, an antibiotic, or a non-metabolizable sugar, optionally wherein the selection agent is glyphosate, glufosinate, mesotrione, isoxaflutole, bicyclopyrone, tembotrione, butafenacil, spectinomycin, bensulfuron-methyl, imazapyr, dicamba, 2,4-D, Haloxyfop, Fluazifop D-xylose, mannose or kanamycin.

16. The method of claim 1, wherein the method further comprises performing an assay on a plant generated from the wounded explant or a sample of the plant to assess for the presence or absence of transformed cells and/or to assess for the number of transformed cells.

17. The method of claim 10, wherein the method further comprises growing the plant to produce a seed and harvesting the seed, wherein the seed optionally comprises at least part of the heterologous polynucleotide.

18. The method of claim 18, wherein the method further comprises growing the seed to produce a progeny plant, optionally wherein the progeny plant comprises at least part of the heterologous polynucleotide.

19. The method of claim 1, wherein the heterologous polynucleotide encodes or comprises a genome editing agent or wherein the heterologous protein comprises a genome editing agent, optionally wherein the genome editing agent is a nuclease or a recombinase.

20. The method of claim 20, wherein the heterologous polynucleotide comprises one or more polynucleotides encoding a Cas protein and/or a guide RNA or wherein the heterologous protein comprises a Cas protein, optionally wherein the Cas protein is Cas9 or Cas12a, or a functional variant thereof.

21. The method of claim 1, wherein the heterologous polynucleotide comprises an expression cassette comprising a coding sequence.

22. The method of claim 22, wherein the expression cassette further comprises a promoter operably linked to the coding sequence.

23. The method of claim 22, wherein the coding sequence encodes a protein or non-coding RNA of interest.

24. The method of claim 1, wherein the contacting in step c) is performed with Agrobacterium, viral particles, microparticles, nanoparticles, cell membrane penetrating peptides, aerosol beam, chemicals, electroporation, or pressure.

25. The method of claim 25, wherein the contacting is performed with Agrobacterium or viral particles and the contacting comprises an infection step and optionally an incubation step.

26. The method of claim 26, wherein the infection step is performed for 30 minutes to 24 hours in darkness and the incubation step is performed for at least 2 days in darkness, optionally 4-5 days.

27. An explant or plant produced by the method of claim 1.

28. A progeny seed produced by crossing the plant of claim 28 with a second plant or by self-crossing the plant of claim 28.

29. A derivative or a commodity product produced or obtained from the plant of claim 28 or a part thereof.

30. A method, comprising:
a) Providing an explant obtained from a seed,
b) Wounding the explant to produce a wounded explant,
c) Contacting the wounded explant with a heterologous polynucleotide comprising a selection marker under conditions where the heterologous polynucleotide enters the wounded explant;
d) Generating a plant from the wounded explant, and
e) Contacting the plant or a part thereof with a selection agent to eliminate or reduce untransformed tissue.

31. A method, comprising:
a) Providing an explant of a seed, wherein the explant comprises an embryo axis and a cotyledon;
b) Contacting the explant with a heterologous polynucleotide and/or heterologous protein under conditions where the heterologous polynucleotide and/or heterologous protein enters the explant.
